(19) United States
(12) Patent Application Publication
(10) Pub. No.: US 2011/0142883 A1
(43) Pub. Date: Jun. 16, 2011

(54) Amorphous Coprecipitates of Atorvastatin Pharmaceutically Acceptable Salts

(75) Inventors: Girish Dixit, Uttar Pradesh (IN); Anil Shahaji Khile, Navi Mumbai (IN); Nitin Sharadchandra Pradhan, Thane (West) (Maharashtra) (IN); Jon Valgeirsson, Hafnarfjordur (IS)

(73) Assignee: ACTAVIS GROUP PTC EHF, Hafnarfjordur (IS)

(21) Appl. No.: 12/669,543

(22) PCT Filed: Jul. 21, 2008
(86) PCT No.: PCT/IB2008/002808

§ 371 Date: Jul. 19, 2010

(30) Foreign Application Priority Data
Jul. 20, 2007 (IN) 1573/CHE/2007
May 20, 2008 (IN) 1241/CHE/2008

(51) Int. Cl. A61K 31/40; A61K 9/14; A61P 9/00; A61P 9/10

(52) U.S. Cl. 424/400; 514/423

(57) ABSTRACT

The present invention relates to stable amorphous co-precipitates of atorvastatin pharmaceutically acceptable salts with pharmaceutically acceptable excipients, method for the preparation, pharmaceutical compositions, and method of treating thereof. Advantageously, the amorphous co-precipitates of atorvastatin pharmaceutically acceptable salts of the present invention have improved physiochemical characteristics that assist in the effective bioavailability

### CROSS REFERENCE TO RELATED APPLICATION

This application claims the benefit of priority to Indian provisional application Nos. 1573/CHE/2007, filed on Jul. 20, 2007, and 1241/CHE/2008, filed on May 20, 2008, which are incorporated herein by reference.

### FIELD OF THE INVENTION

The present invention relates to stable amorphous co-precipitates of atorvastatin pharmaceutically acceptable salts with pharmaceutically acceptable excipients, method for the preparation, pharmaceutical compositions, and method of treating thereof. Advantageously, the amorphous co-precipitates of atorvastatin pharmaceutically acceptable salts of the present invention have improved physiochemical characteristics that assist in the effective bioavailability.

### BACKGROUND OF THE INVENTION

Atorvastatin, chemically known as [R—(R*,R*)]-2-(4-fluorophenyl)-β, δ-dihydroxy-5-(1-methylethyl)-3-phenyl-4-[(phenylamino)carbonyl]-1H-pyrrole-1-heptanoic acid, is an important reductase inhibitor of the enzyme 3-hydroxy-3-methylglutarate-coenzyme A reductase (HMG-CoA) and therefore is a useful anti-hyperlipoproteinemic agent. It has proven to be a highly effective medicament for the treatment of disorders such as hyperlipidemia and hypercholesterolemia which are conditions that are known risk factors for arteriosclerosis and coronary heart disease. Atorvastatin is represented by the following structural formula I:

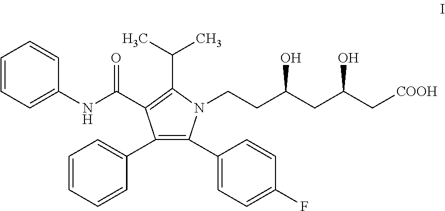

and is marketed as the hemi calcium salt-trihydrate under the name LIPITOR by Warner-Lambert Co. Atorvastatin calcium is used as a lipid-lowering agent for the treatment of hypercholesterolemia and represented by the following structural formula II(a):

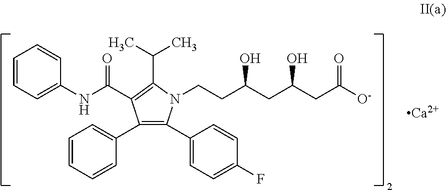

Atorvastatin and its pharmaceutically acceptable salts are disclosed in U.S. Pat. No. 5,273,995 (herein after referred to as the '995 patent). The '995 patent also discloses magnesium salt of atorvastatin and is represented by the following structural formula II(b):

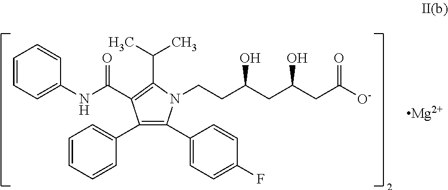

Atorvastatin pharmaceutically acceptable salts in crystalline and amorphous forms, pharmaceutical formulations, and processes for the preparations thereof, are disclosed in U.S. Patent Application No. 2003/0175338; and PCT Application Nos. WO2006/117761, WO2007/118873, WO2007/099552, WO2007/063551, WO2007/132472, and WO2007/057755; which are herein incorporated by reference.

Processes for the preparation of atorvastatin and its salts, atorvastatin lactone, and key intermediates, are disclosed in U.S. Pat. Nos. 5,003,080; 5,097,045; 5,103,024; 5,124,482; 5,149,837; 5,155,251; 5, 216,174; 5,245,047; 5,248,793; 5,280,126; 5,342,952; and 5,397,792. Atorvastatin is usually prepared as the calcium salt since this enables atorvastatin to be conveniently formulated in pharmaceutical formulations, for example, in tablets, capsules, powders and the like for oral administration.

Atorvastatin calcium can exist in amorphous form or in one of several crystalline forms (Form I, Form II, Form III and Form N), which are disclosed in International Publications Nos. WO 97/3958 (U.S. Pat. No. 6,121,461) and WO 97/3959 (U.S. Pat. No. 5,969,156). It is known that the amorphous forms of a number of pharmaceutical substances exhibit different dissolution characteristics and bioavailability patterns compared to the crystalline forms (Konno T., Chem. Pharm. Bull., 1990, 38:2003-2007). For some therapeutic indications the bioavailability is one of the key parameters determining the form of the substance to be used in a pharmaceutical formulation. Since processes for the crystallization and the preparation, respectively, of the amorphous substance are sometimes difficult, and sometimes afford amorphous-crystalline mixtures, that is, a crystalline form instead of an amorphous form, there is a constant need for processes which enable the preparation of a amorphous form without simultaneous formulation of crystalline forms, that is, which will enable the preparation of stable amorphous coprecipitates of atorvastatin pharmaceutically acceptable salts with pharmaceutically acceptable excipients.

Atorvastatin pharmaceutically acceptable salts for example Atorvastatin alkaline earth metal salts such as atorvastatin calcium, atorvastatin magnesium, are substances which are very slightly water-soluble, and it has been found that the crystalline forms are less readily soluble than the amorphous forms, which may cause problems in the bioavailability of atorvastatin in the body. It has been found that the production of amorphous atorvastatin pharmaceutically acceptable salts according to the previously disclosed processes were not consistently reproducible, and therefore a process has been developed for preparing amorphous co-precipitates of atorvastatin pharmaceutically acceptable salts with improved physiochemical characteristics that assist in the effective bioavailability.

The preparation of atorvastatin pharmaceutically acceptable salts produced by the prior art processes also do not have satisfactory purity. Above processes leads to the formation of impurities like O-methyl atorvastatin of formula III, atorvastatin methyl ester of formula IV and increase in level of atorvastatin lactone impurity of formula V, having the following structural formulae:

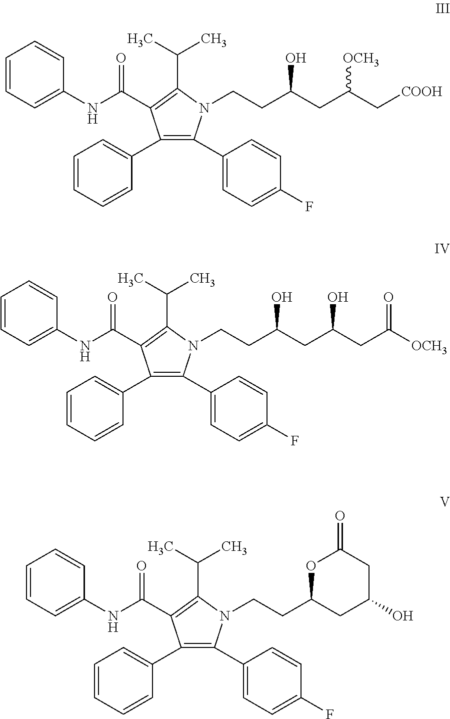

The rate of dissolution of a poorly water-soluble drug is a rate-limiting factor in its absorption by the body. A reduction in the particle size can increase the dissolution rate of such compounds through an increase in the surface area of the solid phase that is in contact with the liquid medium, thereby resulting in an enhanced bioavailability of the compositions containing such compounds. It is generally not possible to predict the exact particle size and distribution required for any particular drug substance to achieve a specific dissolution profile or a specific in vivo behavior, as different drugs show differing dissolution characteristics with a reduction in the particle size.

Particle size reduction beyond certain stage may many times result in other material handling and processing issues such as generation of static charges on new exposed surfaces and agglomeration thereby resulting in unpredictable variations in solubility, dissolution and hence bioavailability. Such problem is addressed in the art by using surface stabilizers to prevent agglomeration.

Thus there is a long-felt need for the development of pharmaceutical compositions of atorvastatin pharmaceutically acceptable salts, preferably alkaline earth metal salts, with improved solubility properties and improved bioavailability characteristics.

### SUMMARY OF THE INVENTION

We have surprisingly found that atorvastatin pharmaceutically acceptable salts form co-precipitates with pharmaceutically acceptable excipients. Such pharmaceutical compositions may be administered easily to a mammalian patient in any dosage form, e.g., liquid, powder, elixir, injectable solution with high rate of bioavailability.

In one aspect, the present invention provides amorphous co-precipitates of atorvastatin pharmaceutically acceptable salts with pharmaceutically acceptable excipients. More particularly, the invention discloses the amorphous co-precipitates of atorvastatin magnesium and atorvastatin calcium with improved physiochemical characteristics which help in the effective bioavailability of atorvastatin magnesium and atorvastatin calcium.

In another aspect, the present invention further encompasses processes for preparing the novel and stable amorphous co-precipitates of atorvastatin pharmaceutically acceptable salts with pharmaceutically acceptable excipients.

The amorphous co-precipitate of atorvastatin pharmaceutically acceptable salts obtained by the processes described in the present invention have improved solubility properties and hence also have improved bioavailability.

In another aspect, the present invention provides highly pure amorphous co-precipitates of atorvastatin pharmaceutically acceptable salts with pharmaceutically acceptable excipients.

In another aspect, the present invention provides the amorphous co-precipitates of atorvastatin pharmaceutically acceptable salts with pharmaceutically acceptable excipients having very low level or free of impurities like O-methyl atovastatin of formula III, atorvastatin methyl ester of formula IV and atorvastatin lactone impurity of formula V.

In another aspect, the present invention provides pharmaceutical compositions comprising the amorphous co-precipitates of atorvastatin pharmaceutically acceptable salts of the present invention and one or more pharmaceutically acceptable excipients.

In still another aspect, the present invention provides pharmaceutical compositions comprising the amorphous co-precipitates of atorvastatin pharmaceutically acceptable salts made by the processes of the present invention, and one or more pharmaceutically acceptable excipients.

In still further aspect, the present invention further encompasses processes for preparing pharmaceutical formulations comprising combining the amorphous co-precipitates of atorvastatin pharmaceutically acceptable salts of the present invention with one or more pharmaceutically acceptable excipients.

### BRIEF DESCRIPTION OF THE DRAWINGS

FIG. 1 is a characteristic powder X-ray diffraction (XRD) pattern of amorphous Atorvastatin magnesium co-precipitate with povidone (K29/32) in weight ratio of 1:1.

FIG. 2 is a characteristic Infra Red (IR) Spectrum of amorphous Atorvastatin magnesium co-precipitate with povidone (K29/32) in weight ratio of 1:1.

FIG. 3 is a characteristic powder X-ray diffraction (XRD) pattern of amorphous

Atorvastatin magnesium co-precipitate with povidone (K29/30).

FIG. 4 is a characteristic Infra Red (IR) Spectrum of amorphous Atorvastatin magnesium co-precipitate with povidone (K29/30).

FIG. 4 is a characteristic Scanning Electron Microscope (SEM) image of the morphological analysis of amorphous Atorvastatin magnesium co-precipitate with povidone (K29/30).

FIG. 6 is a characteristic Scanning Electron Microscope (SEM) image of the morphological analysis of povidone (K29/30).

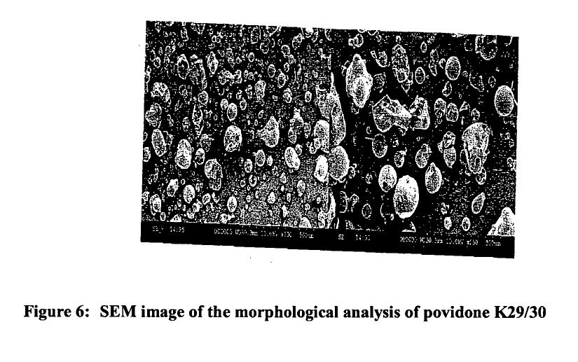

FIG. 7 is a characteristic Scanning Electron Microscope (SEM) image of the morphological analysis of amorphous Atorvastatin magnesium.

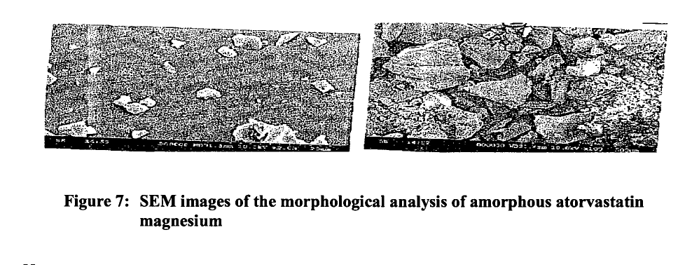

FIG. 8 is a characteristic powder X-ray diffraction (XRD) pattern of amorphous Atorvastatin calcium co-precipitate with povidone (K29/32) in weight ratio of 1:1.

FIG. 9 is a characteristic powder X-ray diffraction (XRD) pattern of amorphous Atorvastatin calcium co-precipitate with povidone (K29/32) in weight ratio of 5:1.

FIG. 10 is a characteristic Infra Red (IR) Spectrum of amorphous Atorvastatin calcium co-precipitate with povidone (K29/32) in weight ratio of 5:1.

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 sec.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 4000 or 3800 to 650 or 450 cm⁻¹.

### DETAILED DESCRIPTION OF THE INVENTION

The term “coprecipitate or co-precipitate” as used herein refers to compositions comprising amorphous atorvastatin pharmaceutically acceptable salt together with at least one pharmaceutically acceptable excipient, being prepared by removing solvent from a solution containing both of them.

The term “pharmaceutical composition” as used herein refers to pharmaceutical formulations comprising amorphous atorvastatin pharmaceutically acceptable salt co-precipitates as described below along with one or more additional pharmaceutically acceptable excipients, as required to formulate the coprecipitates of amorphous atorvastatin pharmaceutically acceptable salt into pharmaceutical compositions for the delivery of atorvastatin magnesium.

According to one aspect of the present invention, there is provided amorphous co-precipitate comprising atorvastatin pharmaceutically acceptable salt and a pharmaceutically acceptable excipient, having improved physiochemical characteristics that assist in the effective bioavailability of atorvastatin pharmaceutically acceptable salts, wherein the pharmaceutically acceptable excipient includes, but are not limited to, pharmaceutical hydrophilic carriers such as polyvinylpyrrolidone (also called povidone), polyvinyl alcohol, hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars, and combinations comprising one or more of the foregoing hydrophilic carriers. Preferable pharmaceutically acceptable excipient is povidone.

In an embodiment, the povidone may be one or more of the grades such as PVP K-15, K-25, K-30, K-60 and K-90, and more preferably PVP K-30.

Preferable pharmaceutically acceptable salts of atorvastatin include alkali and alkaline earth metal salts of atorvastatin such as lithium, sodium, potassium, magnesium, calcium, strontium, and hydrates thereof; more preferably sodium, magnesium, calcium, and hydrates thereof; and still more preferably magnesium, calcium and hydrates thereof.

According to another aspect of the present invention, there is provided pharmaceutical compositions comprising amorphous co-precipitates of atorvastatin pharmaceutically acceptable salts and one or more pharmaceutically acceptable excipient.

The amorphous co-precipitates of atorvastatin pharmaceutically acceptable salts with a pharmaceutically acceptable carrier obtained by the processes of the present invention are characterized by any of their powder X-ray diffraction (XRD) pattern, infrared absorption (IR) spectrum, and SEM images of the morphological analysis.

According to another aspect of the present invention, there is provided amorphous co-precipitates comprising atorvastatin magnesium and a pharmaceutically acceptable carrier, having improved physiochemical characteristics that assist in the effective bioavailability of atorvastatin magnesium.

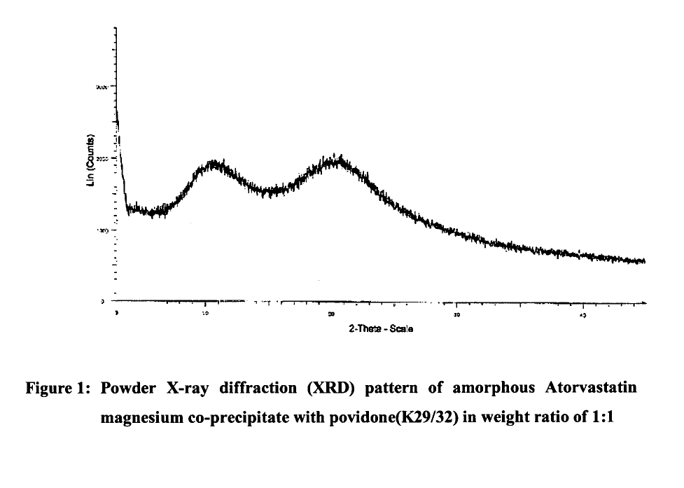

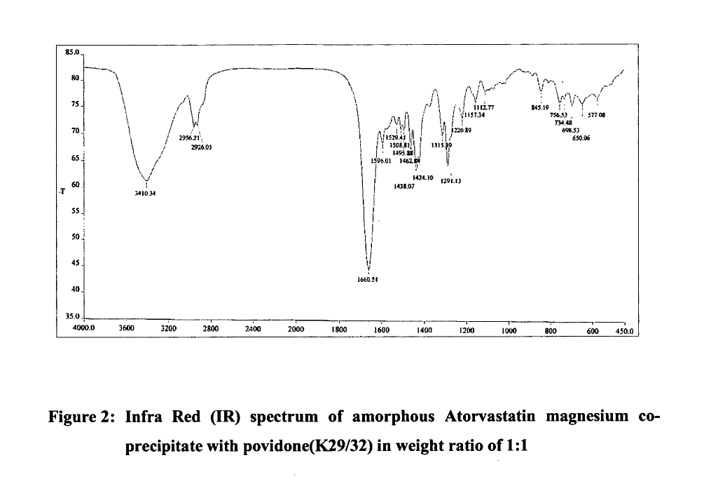

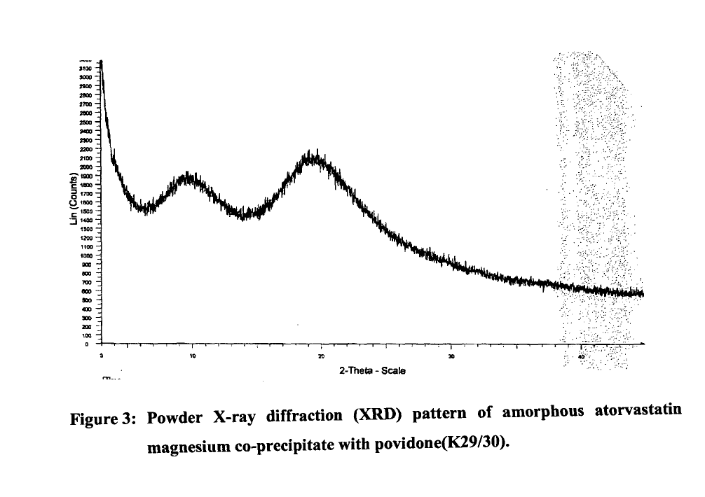

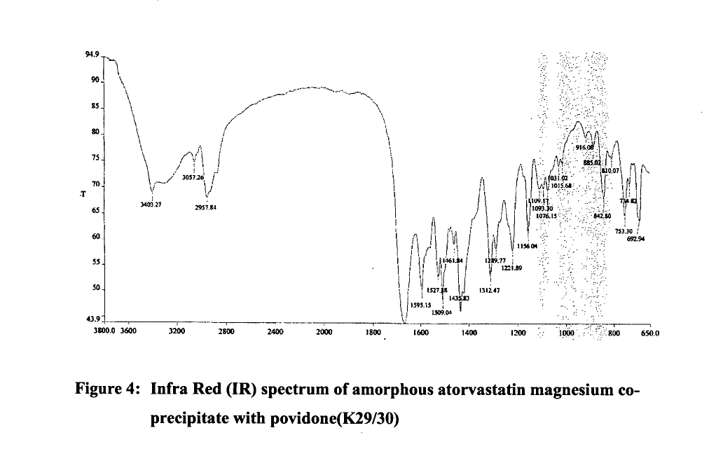

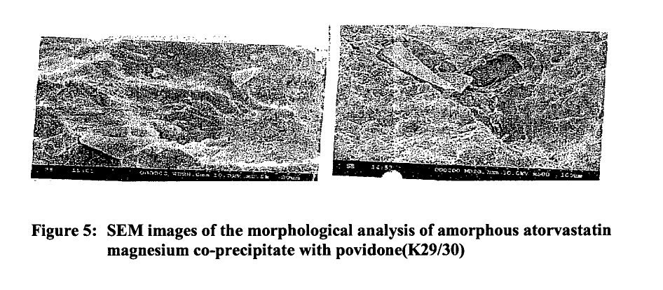

According to another aspect of the present invention, there is provided an amorphous co-precipitate of atorvastatin magnesium with povidone, characterized by at least one, and preferably all, of the following properties:
i) a powder X-ray diffraction pattern substantially in accordance with FIG. 1 or FIG. 3;
ii) an IR spectrum substantially in accordance with FIG. 2 or FIG. 4;
iii) an IR spectrum having absorption bands at about 3410, 2956, 1660, 1596, 1529, 1508, 1462, 1438, 1315, 1291, 1220, 1157, 845, 756 and 698±2 cm⁻¹ substantially as depicted in FIG. 2; or
iv) an IR spectrum having absorption bands at about 3403, 3057, 2957, 1660, 1595, 1527, 1509, 1461, 1435, 1312, 1289, 1221, 1156, 1076, 1031, 842, 753, 692 ±2 cm⁻¹ substantially as depicted in FIG. 4; and
v) a SEM image of the morphological analysis in accordance with FIG. 5.

According to another aspect of the present invention, there is provided amorphous co-precipitates comprising atorvastatin calcium and a pharmaceutically acceptable carrier, having improved physiochemical characteristics that assist in the effective bioavailability of atorvastatin calcium.

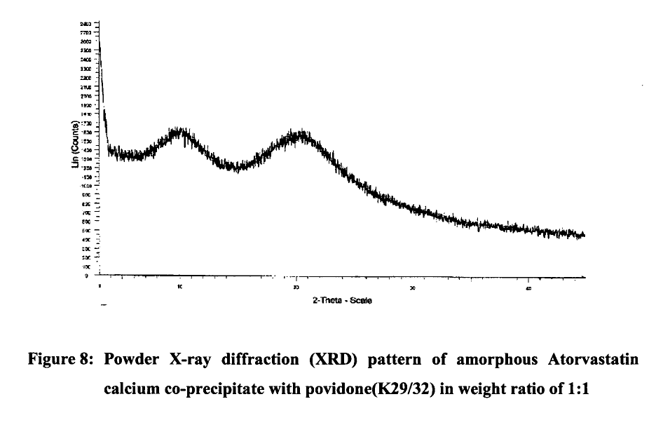

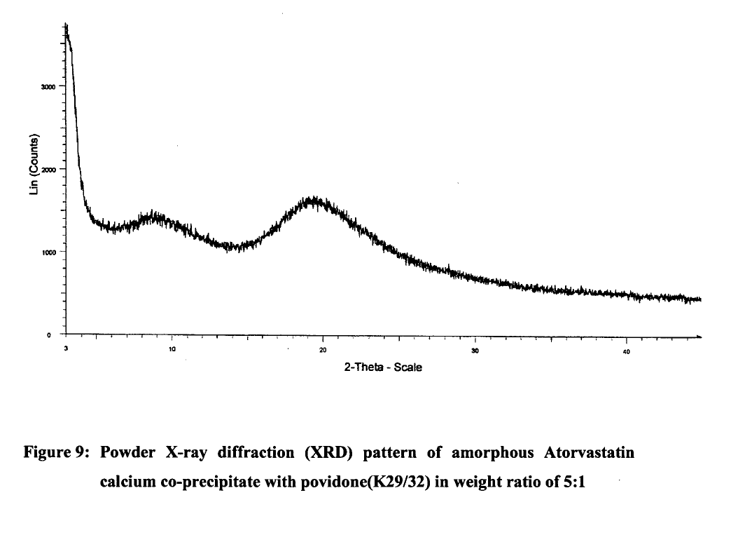

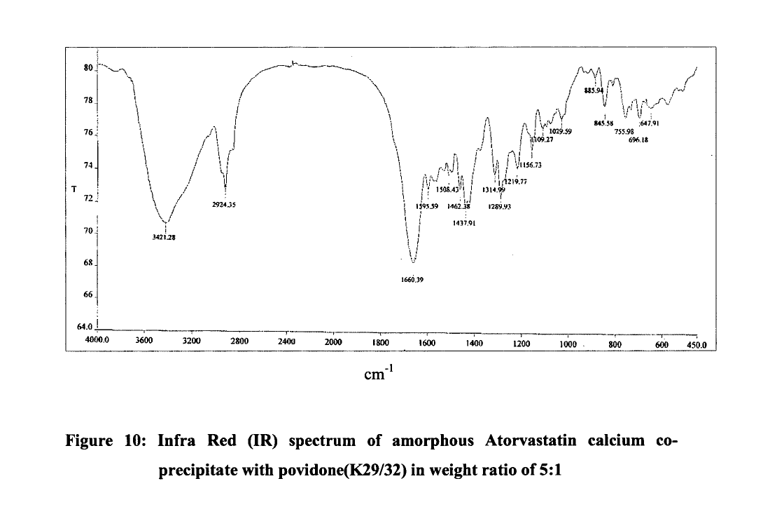

According to another aspect of the present invention, there is provided an amorphous co-precipitate of atorvastatin calcium with povidone, characterized by at least one, and preferably all, of the following properties:
i) a powder X-ray diffraction pattern substantially in accordance with FIG. 8 or FIG. 9;
ii) an IR spectrum substantially in accordance with FIG. 10; and
iii) an IR spectrum having absorption bands at about 3412, 2924, 1660, 1595, 1508, 1462, 1438, 1315, 1290, 1219, 1156, 845, 756 and 696±2 cm⁻¹ substantially as depicted in FIG. 10.

According to another aspect of the present invention, a process for the preparation of amorphous co-precipitate of atorvastatin pharmaceutically acceptable salt with a pharmaceutically acceptable excipient is provided, which comprises the steps of:
a) condensing (4R-cis)-1,1-dimethylethyl 6-(2-aminoethyl)-2,2-dimethyl-1,3-dioxane-4-acetate of formula VI:

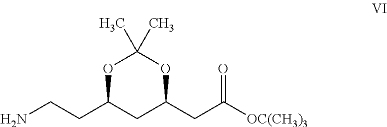

with (±)-4-fluoro-α-(2-methyl-1-oxopropyl)-γ-oxo-N,β-diphenylbenzenebutane amide of formula VII:

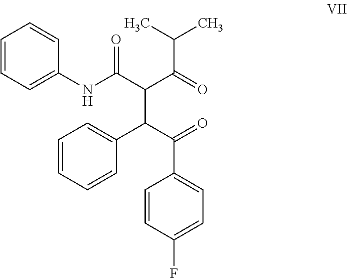

under acidic conditions to produce (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl)-acetic acid tertiary butyl ester of formula VIII:

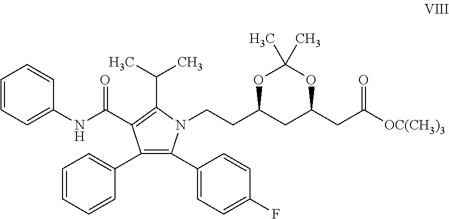

b) contacting the ester compound of formula VIII with an acid for acidic hydrolysis followed by treatment with a suitable base to produce atorvastatin sodium of formula IX:

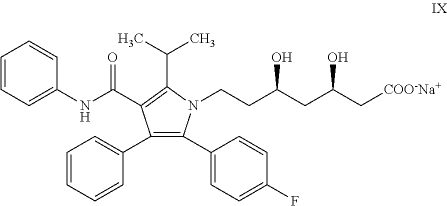

c) converting the atorvastatin sodium of formula IX into a pharmaceutically acceptable alkaline-earth metal salt of atorvastatin of formula II:

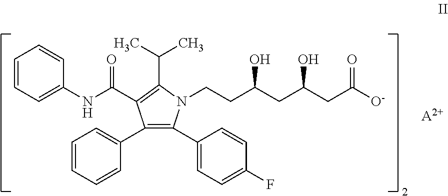

wherein ‘A’ represents an alkaline-earth metal, preferably magnesium and calcium, in water immiscible organic solvent by adding desired metal counter ion;
d) collecting the organic layer containing atorvastatin pharmaceutically acceptable salt obtained in step-(c);
e) removing partially water immiscible organic solvent;
f) combining the reaction mass obtained in step-(e) with a homogeneous solution of a pharmaceutically acceptable excipient;
g) optionally, heating the reaction mixture obtained in step-(f);
h) removing the solvent from the solution obtained in step-(f) or step-(g);
i) optionally, drying the product obtained in step-(h); and
j) recovering amorphous co-precipitate of atorvastatin pharmaceutically acceptable salt with a pharmaceutically acceptable excipient.

The amorphous co-precipitate of atorvastatin pharmaceutically acceptable salt obtained by the process described herein above has improved solubility properties and hence also has improved bioavailability.

In step-(a), the Paal-Knorr condensation of (4R-cis)-1,1-dimethylethyl 6-(2-amino ethyl)-2,2-dimethyl-1,3-dioxane-4-acetate of formula VI with (±)-4-fluoro-α-(2-methyl-1-oxopropyl)-γ-oxo-N,β-diphenyl benzenebutane amide of formula VII was found to work efficiently in organic solvents, which solvents are important for water removal and solubility of the reactants. Optimal solvent combinations for the improved process are mixtures of cyclohexane: toluene: tetrahydrofuran. However, solvents may be selected from toluene or other aromatic hydrocarbons, tetrahydrofuran, hexane, heptane, cyclohexane, and other acyclic and cyclic aliphatic hydrocarbons. Binary mixtures of the above may also be used such as 1,4-dioxane:methyltert-butyl ether, 1,4-dioxane:hexane, 1,4-dioxane:methylcyclohexane, 1,4-dioxane:cyclohexane, dimethyl ether:hexane or mixture thereof. The reaction may be carried out at boiling temperature of reaction solvent. The reaction time may vary from about 20 to 60 hours, more preferably 45 to 55 hours. The reaction can be carried out with or without removing water azeotropically. The product may be isolated by methods known in the art. The product may be further purified or may be directly used for next step.

The step-(b) can be carried out in alcoholic or nitrile or cyclic ether solvents. Suitable alcohol solvents include, but are not limited to, aromatic and aliphatic C₁-C₁₂ alcohols and the like and mixtures thereof. Suitable aliphatic alcohols include C₁-C₈ alcohols such as, for example, methanol, ethanol, n-propanol, isopropanol, n-butanol, isobutanol, tert-butanol and the like and mixtures thereof. Suitable aromatic alcohols include C₃-C₁₂ alcohols such as, for example, benzyl alcohol, benzyloxyethanol, phenoxyethanol and the like and mixtures thereof. Suitable nitrile solvent is acetonitrile. Suitable cyclic ether solvents include, but are not limited to, tetrahydrofuran, 1,4-dioxan, pyran and the like and mixtures thereof. Most preferable alcoholic solvent is isopropanol. The reaction may be carried out at a temperature of about 25° C. to the reflux temperature of the solvent used. The reaction time may vary from about 4 to 16 hours, and more preferably 5 to 12 hours. The product may be isolated by methods known in the art. The product may be further purified or may be directly used for next step.

In step-(c), the atorvastatin sodium obtained in step-(b) can be converted to an appropriate pharmaceutically acceptable salt of atorvastatin such as magnesium and calcium salts. In an embodiment, atorvastatin magnesium is prepared by treating atorvastatin sodium with an aqueous solution of magnesium hydroxide or a magnesium salt such as magnesium acetate, magnesium chloride, magnesium sulphate, magnesium nitrate and magnesium phosphate. In another embodiment, atorvastatin calcium is prepared by treating atorvastatin sodium with a suitable calcium salt. The reaction may be performed at a temperature of below about 50° C. for at least 15 minutes, and more preferably at a temperature of about 15° C. to about 35° C. from about 30 minutes to about 2 hours. The suitable calcium salts include organic and inorganic salts of calcium which are capable of dissociating into Ca²⁺ and an anionic component when added to the atorvastatin salt solution. Among the organic salts that may be used are carboxylates and sulfonates. Exemplary carboxylates are lower alkyl carboxylates such as acetate, proprionate, butyrate and tartrate; aryl carboxylates such as benzoate and phthalate; and higher alkyl carboxylates such as stearate, dodecanoate and the like. Also included are calcium ascorbate and succinate. Among the sulfonates that may be used are lower alkyl and aryl sulfonates like calcium methane sulfonate, calcium benzene sulfonate and calcium p-toluene sulfonate. The preferred organic calcium salts are lower carboxylate salts, and the most preferred organic calcium salt is calcium acetate. Depending upon solubility, inorganic calcium salts which may be used include halide salts such as CaCl₂, CaF₂, CaBr₂ and CaI₂, as well as calcium borate (B₄CaO₇), calcium tetrafluoroborate (CaBF₄), calcium carbonate (CaCO₃), monobasic calcium phosphate (Ca(H₂PO₄)₂), dibasic calcium phosphate (CaHPO₄) and tribasic calcium phosphate (Ca(PO₄)₂), calcium sulfate (CaSO₄) and calcium hydroxide (Ca(OH)₂), and hydrates thereof.

The atorvastatin sodium may optionally be treated with a suitable acid prior to treatment with an appropriate metal counter ion. The aqueous solution of metal counter ion can be added in presence of water-immiscible or slightly water-miscible solvents to extract pharmaceutically acceptable atorvastatin magnesium. Suitable solvents for the extractions include ethers such as tetrahydrofuran, 1,4-dioxan, esters such as ethyl acetate and isopropyl acetate, hydrocarbons such as toluene and xylene.

Preferably, the calcium salt may be treated with the atorvastatin salt solution by adding the calcium salt in substantially pure form, i.e. either as a solid or, if liquid, as a neat liquid, to the atorvastatin salt solution or, more preferably, by first forming a calcium salt solution and then combining the atorvastatin salt solution with calcium salt solution. It is most preferred to combine the calcium salt and the atorvastatin salt solution by first dissolving the calcium salt in a solvent and then adding the calcium salt solution to the atorvastatin salt solution slowly. The preferred calcium salt is calcium acetate and the preferred calcium salt solvent is water.

Step-(e) involves partial removal of the solvent from the solution obtained from step-(d) using a suitable technique. Removal of the solvent may be carried out suitably using techniques such as evaporation, atmospheric distillation, or distillation under vacuum. The distillation of the solvent may be conducted under a vacuum such as below about 100 mm Hg to below about 600 mm Hg, and at elevated temperatures such as about 20° C. to about 70° C. Any temperature and vacuum conditions can be used as long as there is no increase in the impurity levels of the product due to decomposition.

In step-(f), the pharmaceutical excipient can be dissolved in a solution containing atorvastatin pharmaceutically acceptable salt or atorvastatin pharmaceutically acceptable salt can be dissolved in a solution containing a pharmaceutical excipient.

Alternatively, a solution containing atorvastatin pharmaceutically acceptable salt can be combined with a solution containing a pharmaceutical excipient, and the solvents used for preparing the different solutions need not be the same as long as the solvents have mutual solubility and form a single phase. In any event, atorvastatin pharmaceutically acceptable salt must be completely soluble in the solvents used and should provide a clear solution. The presence of undissolved crystals could lead to the formation of a material that is not completely amorphous.

Suitable organic solvents that can be used for dissolving atorvastatin pharmaceutically acceptable salt either alone or along with a pharmaceutically acceptable excipient include, but are not limited to, alcohols such as methanol, ethanol, isopropyl alcohol, n-propanol and the like, and mixtures thereof; halogenated hydrocarbons such as dichloromethane, 1,2-dichloroethane, chloroform, carbon tetrachloride and the like, and mixtures thereof; ketones such as acetone, ethyl methyl ketone, methyl isobutyl ketone and the like, and mixtures thereof; esters such as ethyl acetate, n-propyl acetate, n-butyl acetate, t-butyl acetate and the like, and mixtures thereof; hydrocarbons such as toluene, xylene and the like, and mixtures thereof; nitriles such as acetonitrile, propionitrile and the like, and mixtures thereof; or combinations comprising one or more of the foregoing solvents.

The pharmaceutically acceptable excipient that can be used for the preparation of co-precipitates with atorvastatin pharmaceutically acceptable salts include, but are not limited to, pharmaceutical hydrophilic carriers such as polyvinylpyrrolidone (also called povidone), polyvinyl alcohol, hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars, and combinations comprising one or more of the foregoing hydrophilic carriers. Preferable pharmaceutically acceptable excipient is povidone. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

In an embodiment, the povidone may be used in one or more of the grades such as PVP K-15, K-25, K-30, K-60 and K-90, and more preferably PVP K-30.

These lists of solvents and pharmaceutically acceptable carriers are merely representative of those that can be used, and the lists are not intended to be exhaustive or limiting. Generally, the more volatile solvents are preferred to reduce the energy requirements for subsequent solvent removal.

The quantity of solvent used for dissolution depends on the solvent and the dissolution temperature adopted. The concentration of atorvastatin pharmaceutically acceptable salt in the solution may generally range from about 0.05 to about 0.1 g/ml in the solvent. In general, the volumes will be kept to a minimum, to facilitate the eventual solvent removal.

The heating in step-(g) is optionally carried out at a temperature of about 30° C. to the reflux temperature of the solvent used for at least 20 minutes, and preferably at a temperature of about 40° C. to about 75° C. from about 30 minutes to about 4 hours.

The solution obtained in step-(f) or step-(g) can optionally be treated with materials such as carbon for clarification or with sodium sulfate for moisture removal. Optionally, the solution obtained above can be treated to remove the undissolved particles, prior to further process steps. Any undissolved particles can be removed suitably by filtration, centrifugation, decantation, and other techniques. The solution can be filtered by passing through paper, glass fiber, or other membrane material, or a particulate filtration medium such as celite or hyflow. Depending upon the equipment used and the solution properties, such as concentration and temperature of the solution, the filtration apparatus may need to be preheated to avoid crystallization.

Step-(h) involves the removal of solvent from the solution obtained in step-(f) or step-(g), using a suitable technique. Removal of the solvent may be carried out suitably using techniques such as evaporation, atmospheric distillation, or distillation under vacuum.

Distillation of the solvent may be conducted under a vacuum such as below about 100 mm Hg to below about 600 mm Hg, and at elevated temperatures such as about 20° C. to about 70° C. Any temperature and vacuum conditions can be used as long as there is no increase in the impurity levels of the product due to decomposition.

Suitable techniques which can be used for the distillation include, without limitation thereto, distillation using a rotational evaporator device such as a Buchi Rotavapor, spray drying, agitated thin film drying (“ATFD”), and the like. Generally, techniques providing a rapid solvent removal will be utilized to provide the desired amorphous form of atorvastatin magnesium with pharmaceutically acceptable excipients.

The amorphous material obtained from step-(h) can be collected from the equipment using techniques such as by scraping the container. Other product collection techniques will be used for spray drying, and are well known in the art.

Step-(i) involves an optional drying of the product obtained from step-(h) to afford the amorphous atorvastatin pharmaceutically acceptable salt co-precipitate with pharmaceutically acceptable excipients, substantially free of residual solvents.

Drying can be carried out under reduced pressure until the residual solvent content reduces to the desired amount, such as an amount that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm. The drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance.

Drying can be suitably carried out in a tray dryer, vacuum oven, air oven, or using a fluidized bed drier, spin flash dryer, flash dryer and the like. Drying equipment selection is well within the ordinary skill in the art.

According to another aspect of the present invention, a process for preparing amorphous co-precipitates of atorvastatin pharmaceutically acceptable salts and a pharmaceutically acceptable excipient is provided, which comprises the steps of:
a) providing a solution of atorvastatin pharmaceutically acceptable salt and a suitable pharmaceutically acceptable excipient in a suitable organic solvent;
b) optionally, filtering the solvent solution to remove any extraneous matter; and
c) substantially removing the solvent from the solution to afford amorphous co-precipitates of atorvastatin pharmaceutically acceptable salt with pharmaceutically acceptable excipient.

The process can produce amorphous co-precipitates of atorvastatin pharmaceutically acceptable salts with pharmaceutically acceptable excipient in substantially pure form.

The term “substantially pure amorphous atorvastatin pharmaceutically acceptable salt co-precipitate with pharmaceutically acceptable excipient” refers to the amorphous atorvastatin pharmaceutically acceptable salt co-precipitate having purity greater than about 98%, specifically greater than about 99%, more specifically greater than about 99.5% and still more specifically greater than about 99.9% (measured by HPLC).

The amorphous atorvastatin pharmaceutically acceptable salt co-precipitates obtained by the processes disclosed herein above are stable, consistently reproducible and have good flow properties, and which is particularly suitable for bulk preparation and handling, and so, the novel co-precipitates obtained by the process disclosed herein are suitable for formulating atorvastatin pharmaceutically acceptable salts.

Preferable pharmaceutically acceptable salts of atorvastatin include alkali and alkaline earth metal salts of atorvastatin such as lithium, sodium, potassium, magnesium, calcium, strontium; more preferably sodium, magnesium and calcium; and still more preferably magnesium and calcium.

The pharmaceutically acceptable excipient that can be used for the preparation of co-precipitates with atorvastatin pharmaceutically acceptable salts include, but are not limited to, pharmaceutical hydrophilic carriers such as polyvinylpyrrolidone (also called povidone), polyvinyl alcohol, hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars, and combinations comprising one or more of the foregoing hydrophilic carriers. Preferable pharmaceutically acceptable excipient is povidone. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

In an embodiment, the povidone may be used in one or more of the grades such as PVP K-15, K-25, K-30, K-60 and K-90, and more preferably PVP K-30.

Step-(a) of providing a solution of atorvastatin pharmaceutically acceptable salt includes dissolving any form of atorvastatin pharmaceutically acceptable salt in an organic solvent, or such a solution may be obtained directly from a reaction in which atorvastatin pharmaceutically acceptable salts are formed. The pharmaceutical excipient can be dissolved in a solution containing atorvastatin pharmaceutically acceptable salt or atorvastatin pharmaceutically acceptable salt can be dissolved in a solution containing a pharmaceutical excipient.

Alternatively, a solution containing pharmaceutically acceptable salt of atorvastatin can be combined with a solution containing a pharmaceutically acceptable excipient, and the solvents used for preparing the different solutions need not be the same as long as the solvents have mutual solubility and form a single phase. In any event, atorvastatin pharmaceutically acceptable salt must be completely soluble in the solvents used and should provide a clear solution. The presence of undissolved crystals could lead to the formation of a material that is not completely amorphous.

Suitable organic solvents that can be used for dissolving pharmaceutically acceptable salt of atorvastatin either alone or along with a pharmaceutically acceptable excipient include, but are not limited to, alcohols such as methanol, ethanol, isopropyl alcohol, n-propanol and the like, and mixtures thereof; halogenated hydrocarbons such as dichloromethane, 1,2-dichloroethane, chloroform, carbon tetrachloride and the like, and mixtures thereof; ketones such as acetone, ethyl methyl ketone, methyl isobutyl ketone and the like, and mixtures thereof; esters such as ethyl acetate, n-propyl acetate, n-butyl acetate, t-butyl acetate and the like, and mixtures thereof; hydrocarbons such as toluene, xylene and the like, and mixtures thereof; nitriles such as acetonitrile, propionitrile and the like, and mixtures thereof; or combinations comprising one or more of the foregoing solvents.

These lists of solvents and pharmaceutically acceptable carriers are merely representative of those that can be used, and the lists are not intended to be exhaustive or limiting. Generally, the more volatile solvents are preferred to reduce the energy requirements for subsequent solvent removal.

Preferably the dissolution is carried out at a temperature of about 20° C. to about 140° C., more preferably at about 25° C. to about 100° C., and still more preferably at about 40° C. to about 80° C.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing atorvastatin pharmaceutically acceptable salt and appropriate pharmaceutically acceptable excipient by removing charcoal. Preferably, finely powdered carbon is an active carbon.

The solution obtained in step-(a) or step-(b) is optionally stirred at a temperature of about 30° C. to the reflux temperature of the solvent used for at least 20 minutes, and preferably at a temperature of about 40° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilization or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

One of the preferred methodologies to remove the solvent involves spray-drying, in which a solution of atorvastatin calcium is sprayed into the spray drier at the flow rate ranging from 10 to 300 ml/hr, preferably flow rate is 40 to 200 ml/hr. The air inlet temperature to the spray drier used may range from 25 to 150° C., preferably from 60° C. to 110° C. and the outlet air temperature used may range from 30 to 90° C. Another preferred method is vertical agitated thin-film drying (or evaporation). Agitated thin film evaporation technology involves separating the volatile component using indirect heat transfer coupled with mechanical agitation of the flowing film under controlled condition.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The substantially pure amorphous co-precipitates of atorvastatin pharmaceutically acceptable salt with pharmaceutically acceptable excipient obtained by the above processes may be further dried in, for example, Vacuum Tray Dryer, Rotocon Vacuum Dryer, Vacuum Paddle Dryer or pilot plant Rota vapor, to further lower residual solvents.

The total purity of the amorphous co-precipitates of atorvastatin pharmaceutically acceptable salt with pharmaceutically acceptable excipient obtained by the processes disclosed herein above is of greater than about 99.9%, specifically greater than about 99.95%, and more specifically greater than about 99.99% as measured by HPLC.

The dried product obtained by the processes disclosed herein above can optionally be milled to get desired particle sizes. Milling or micronization can be performed prior to drying, or after the completion of drying of the product. The milling operation reduces the size of particles and increases surface area of particles. Drying is more efficient when the particle size of the material is smaller and the surface area is higher, hence milling will frequently be performed prior to the drying operation.

Milling can be done suitably using jet milling equipment like an air jet mill, or using other conventional milling equipment.

The resulting amorphous powder compositions of the invention have improved solubility properties and hence also have improved bioavailability.

In accordance with another embodiment of the present invention, highly pure amorphous atorvastatin pharmaceutically acceptable salt co-precipitate with pharmaceutically acceptable excipients is provided.

In accordance with another embodiment of the present invention, co-precipitate of amorphous atorvastatin pharmaceutically acceptable salts and pharmaceutically acceptable excipients having very low level or substantially free of impurities like O-methyl atovastatin of formula III, atorvastatin methyl ester of formula IV and atorvastatin lactone impurity of formula V is provided.

The amorphous co-precipitates of atorvastatin pharmaceutically acceptable salts with the pharmaceutically acceptable excipients obtained by the present process are a random distribution of the atorvastatin pharmaceutically acceptable salt and the pharmaceutically acceptable excipient in a particle matrix. While the invention should not be constrained by any particular theory, the coprecipitates have the characteristics of solid dispersions at a molecular level, being in the nature of solid solutions. The solid solutions, or molecular dispersions, provide homogeneous particles in which no discrete areas of only amorphous atorvastatin pharmaceutically acceptable salt and only pharmaceutically acceptable excipient can be observed.

In one embodiment, the co-precipitate of amorphous atorvastatin pharmaceutically acceptable salt with a pharmaceutically acceptable excipient disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

In another embodiment, the particle sizes of the amorphous atorvastatin pharmaceutically acceptable salt co-precipitate with a pharmaceutically acceptable excipient can be achieved via comminution, or a mechanical process of reducing the size of particles which includes any one or more of cutting, chipping, crushing, milling, grinding, micronizing, trituration or other particle size reduction methods known in the art, to bring the solid state forms the desired particle size range.

As used herein, Dₓ means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

According to another aspect of the present invention, there is provided a method for treating or preventing cardiovascular diseases caused by hypercholesterolemia, hyperlipoproteinemia, and atherosclerosis, comprising administering the amorphous atorvastatin pharmaceutically acceptable salt co-precipitate, or a pharmaceutical composition that comprises amorphous atorvastatin pharmaceutically acceptable salt co-prepcipitate, along with pharmaceutically acceptable excipients.

According to another aspect of the present invention, there is provided pharmaceutical compositions comprising the amorphous atorvastatin pharmaceutically acceptable salt co-precipitate and one or more pharmaceutically acceptable excipients.

According to another aspect of the present invention, there is provided a process for preparing a pharmaceutical formulation comprising combining amorphous atorvastatin pharmaceutically acceptable salt co-precipitate with one or more pharmaceutically acceptable excipients.

The powder compositions of this invention as described in the different embodiments above are useful in the preparation of pharmaceutical compositions for the delivery of atorvastatin pharmaceutically acceptable salt. As used herein, pharmaceutical composition means a composition (medicament) for use in treating a mammal that includes atorvastatin pharmaceutically acceptable salt and is prepared in a manner that is appropriate for administration to a mammal, such as a human. A pharmaceutical composition contains one or more pharmaceutically acceptable excipients that are non-toxic to the mammal intended to be treated when the composition is administered in an amount effective to treat the mammal.

The pharmaceutical compositions may be in the form of encapsulated free flowing powders or granules; compressed solid dosage forms such as tablets like chewable or dispersible or mouth dissolving; pellets (extruded or fluidized) or beads or spheres or cores (water-soluble or insoluble or both) filled into sachets or capsules; enterable solutions, syrup, suspensions or dispersions; emulsions like micro-emulsions or multiple-emulsions; elixirs, troches, lozenges, lyophilized powders and the like.

Also the lyophilized powders or enteric solutions or suspensions or dispersions, emulsions like micro-emulsions or multiple-emulsions, of atorvastatin pharmaceutically acceptable salt can further be filled into hard or soft gelatin capsules.

The pharmaceutical compositions of the present invention may contain one or more diluents to make up the final composition mass so that it becomes easier for the patient and the caregiver to handle. Common diluents that can be used in pharmaceutical formulations comprise microcrystalline cellulose (MCC), silicified MCC (e.g. Prosolv™ HD 90), micro fine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like.

The pharmaceutical compositions may further include a disintegrant. Useful disintegrants include but not limited to methyl cellulose, microcrystalline cellulose, carboxymethyl cellulose calcium, carboxymethyl cellulose sodium (e.g. Ac-Di-Sol®, Primellose®), crospovidone (e.g. Kollidon®, Polyplasdone®), povidone K-30, guar gum, magnesium aluminum silicate, colloidal silicon dioxide (Aerosil®), polacrilin potassium, starch, pregelatinized starch, sodium starch glycolate (e.g. Explotab®), and sodium alginate.

Pharmaceutical compositions may further include ingredients such as, but are not limited to, pharmaceutically acceptable glidants, lubricants, opacifiers, colorants, sweeteners, thickeners and other commonly used excipients.

In the following section preferred embodiments are described by way of examples to illustrate the process of this invention. However, these are not intended in any way to limit the scope of the present invention.

### EXAMPLE 1

### Preparation of (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tertiary butyl ester

### Step-I: Preparation of Crude Product

A mixture of (±)-4-fluoro-α-(2-methyl-1-oxopropyl)-γ-oxo-N,β-diphenyl benzenebutane amide (250.0 gm), (4R-cis)-1,1-dimethylethyl 6-(2-aminoethyl)-2,2-dimethyl-1,3-dioxane-4-acetate (188.0 gm), cyclohexane (875.0 ml), toluene (187.5 ml), tetrahydrofuran (187.5 ml) and pivalic acid (40.0 gm) were heated to reflux temperature and maintained at reflux temperature (70-80° C.) with azeotropic water removal for about 36 to 40 hours. The reaction was monitored by HPLC. After completion reaction (starting material content: less than 5.0%), the reaction mass was cooled to 25-30° C. The reaction mass was charcoalized with activated charcoal (12.5 gm) for 1 hour at 25-30° C. followed by filtration to remove charcoal from solution through hyflow supercel bed and washed the bed with toluene (250 ml). The combined washing and filtrate was concentrated under vacuum and solvent exchange was performed using isopropyl alcohol (33 250.0 ml) and the reaction mass was concentrated. Isopropyl alcohol (1100.0 ml) was added to the concentrated mass and heated at 80-85° C. to obtain clear solution. The resulting solution was cooled gradually to 20-25° C. (approximately 2 to 3 hours) and stirred at this temperature for 10 to 12 hours. Next, the resulting mass was cooled to 0-5° C. and stirred for 3 hours at this temperature. The resulting solid was filtered and washed with chilled isopropyl alcohol (2×250.0 ml) and spin dried to give wet crude (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tertiary butyl ester.

### Step-II: Purification of Crude Product

The wet crude product (obtained above) was suspended in isopropyl alcohol (1100.0 ml) and heated at 80-85° C. to obtain clear solution. The resulting solution was cooled gradually to 20-25° C. (approximately 2 to 3 hours) and stirred at this temperature for 10 to 12 hours. Next, the resulting mass was cooled to 0-5° C. and stirred for 3 hours at this temperature. The resulted solid was filtered and washed with chilled isopropyl alcohol (2×250.0 ml) followed by cyclohexane (250.0 ml).The wet product was spin dried and further dried under reduced pressure to give dry pure (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1 3]-dioxane-4-yl-acetic acid tertiary butyl ester. (Yield=262.50 gm, HPLC purity: 99.75%).

### EXAMPLE 2

### Preparation of Atorvastatin sodium

(4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tertiary butyl ester (200.0 gm) was suspended in isopropanol (2800.0 ml) under stirring. Next, diluted hydrochloric acid solution (1:1, 100.0 ml) was added at 25-30° C. in 30 minutes. The reaction mixture was heated at 40-45° C. and stirred for 4 to 5 hours at 40-45° C. The reaction was monitored by HPLC. After completion of the reaction, the mass was cooled to 25-30° C. The resulting reaction mass was added to another reaction assembly containing 23.0% w/w sodium hydroxide solution (148.0 gm) maintaining temperature between 25-30° C. The resulting mass was further stirred for 12 hours at 25-30° C. and maintaining pH 13.5 to 14.50. The pH of reaction mass was maintained between 13.5 and 14.5 by adding 23% w/w sodium hydroxide solution through out reaction. The reaction was monitored by HPLC. After completion of reaction, the solid thus obtained was further stirred for 2 hours and filtered, washed with isopropyl alcohol (2×200.0 ml), cyclohexane (616.0 ml) and dried at 50-55° C. to give pure atorvastatin sodium (194.0 gm, HPLC purity: 99.65%).

### EXAMPLE 3

### Preparation of Amorphous Coprecipitate of Atorvastatin Magnesium with Povidone in a Ratio of (1:1)

2.0 grams of atorvastatin magnesium and 2.0 grams of povidone USP (PVP K29/32) were suspended in 300 ml of dichloromethane and refluxed at 40 to 45° C. to get clear solution. The solution was filtered in hot conditions and dichloromethane was distilled in a Buchi Rotavapor apparatus under a vacuum to afford dried coprecipitate of atorvastatin magnesium with povidone. Yield =3.60 grams

### EXAMPLE 4

### Preparation of Amorphous Coprecipitate of Atorvastatin Magnesium with Povidone in a Ratio of (1:1)

Atorvastatin sodium (25 g, prepared as per Example 2) was suspended in ethyl acetate (150 ml) and stirred for 5 minutes. Next, aqueous magnesium acetate tetra hydrate solution (prepared by dissolving 6 gm of magnesium acetate tetra hydrate in 150 ml purified water) was added. The resulting mass was further stirred for 30 minutes at 25 to 30° C. followed by layer separation. Aqueous layer was further extracted with ethyl acetate (50 ml). The combined ethyl acetate layer was washed with 15% aqueous sodium chloride solution (100 ml). The ethyl acetate layer was dried over sodium sulfate and concentrated under vacuum at 40 to 45° C. Next, methanolic povidone USP (K29/32) solution (prepared by dissolving 25 g povidone USP in 150 ml methanol) was added and the resulted reaction mixture was stirred for 15 minutes. The clear solution was concentrated in a Buchi Rotavapor apparatus under a vacuum. 42.0 g of a dried coprecipitate of atorvastatin magnesium with povidone was obtained.

### EXAMPLE 5

### Preparation of Amorphous Coprecipitate of Atorvastatin Magnesium with Povidone in a Ratio of (1:1)

Atorvastatin sodium (12.5 g, prepared as per Example 2) was suspended in ethyl acetate (75 ml) and stirred for 5 minutes. Next, aqueous magnesium acetate tetrahydrate solution (prepared by dissolving 3 gm of magnesium acetate tetrahydrate in 75 ml purified water) was added. The resulting mass was further stirred for 30 minute at 25 to 30° C. followed by layer separation. Aqueous layer was further extracted with ethyl acetate (50 ml). The combined ethyl acetate layer was washed with 15% aqueous sodium chloride solution (50 ml). The ethyl acetate layer was dried over sodium sulfate and concentrated under vacuum at 40 to 45° C. Next, methanol (100 ml) was added to the reaction mass followed by the addition of methanolic povidone USP (K29/32) solution (prepared by dissolving 12.5 gm povidone USP in 75 ml methanol). The resulted mass was further stirred for 15 minutes. The clear solution was concentrated to dryness using laboratory spray dryer (Jay Instruments & Systems Pvt. Ltd. India, Model-LSD-48 mini Spray Dryer). 8.5 g of a dried coprecipitate of atorvastatin magnesium with povidone was obtained.

### EXAMPLE 6

### Preparation of Amorphous Coprecipitate of Atorvastatin Calcium with Povidone in a Ratio of (1:1)

Atorvastatin calcium (1.0 g) and 1.0 g of povidone USP (PVP K29/32) were dissolved in methanol (60 ml) and stirred at 25 to 30° C. to get a clear solution. The clear solution was filtered and methanol was distilled in a Buchi Rotavapor apparatus under a vacuum.1.83 g of a dried coprecipitate of atorvastatin calcium with povidone was obtained.

### EXAMPLE 7

### Preparation of Amorphous Co-Precipitate of Atorvastatin Magnesium with Povidone (k29/30)

Atorvastatin sodium (50 g) was suspended in ethyl acetate (300 ml) and stirred for 5 minutes. Next, aqueous magnesium acetate tetra hydrate solution (prepared by dissolving 12.0 gm of magnesium acetate tetra hydrate in 300 ml purified water) was added. The resulting mass was further stirred for 30 minutes at 25-30° C. followed by layer separation. Aqueous layer was further extracted with ethyl acetate (2×50 ml). The combined ethyl acetate layer was washed with 15% aqueous sodium chloride solution (200 ml). The ethyl acetate layer was dried over sodium sulfate and concentrated under vacuum at 40-45° C. Next, methanolic povidone USP (K29/32) solution (prepared by dissolving 11.2 g povidone USP in 112 ml methanol) was added and the resulted reaction mixture was stirred for 15 minutes. The clear solution was concentrated in a Buchi Rotavapor apparatus under a vacuum. 48.8 g of a dried co-precipitate of atorvastatin magnesium with povidone was obtained.

### EXAMPLE 8

### Preparation of Amorphous Coprecipitate of Atorvastatin Calcium with Povidone (5:1)

Atorvastatin sodium (5 g, prepared as per Example 2) was suspended in ethyl acetate (30 ml) and stirred for 5 minutes. Next, aqueous calcium acetate dihydrate solution (prepared by dissolving 0.88 gm of calcium acetate dihydrate in 30 ml purified water) was added. The resulting mass was further stirred for 30 minutes at 25 to 30° C. followed by the layer separation. The aqueous layer was further extracted with ethyl acetate (10 ml). The combined ethyl acetate layer was washed with 15% aqueous sodium chloride solution (20 ml) and dried over sodium sulfate followed by concentration under vacuum at 40 to 45° C. Next, methanolic povidone USP (K29/32) solution (prepared by dissolving 1 gm povidone USP in 5 ml methanol) was added the resulted reaction mixture was stirred for 15 minutes. The clear solution was concentrated to dryness in a Buchi Rotavapor apparatus under a vacuum. 4.35 g of a dried coprecipitate of atorvastatin calcium with povidone was obtained.

### EXAMPLE 9

### Preparation of Amorphous Coprecipitate of Atorvastatin Calcium with Povidone in a Ratio of (5:1)

Atorvastatin sodium (5 g, prepared as per Example 2) was suspended in ethyl acetate (30 ml) and stirred for 5 minutes. Next, aqueous calcium acetate dihydrate solution (prepared by dissolving 0.88 gm of calcium acetate dihydrate in 30 ml purified water) was added. The resulting mass was further stirred for 30 minutes at 25 to 30° C. followed by layer separation. The aqueous layer was further extracted with ethyl acetate (10 ml). The combined ethyl acetate layer was washed with 15% aqueous sodium chloride solution (20 ml). The ethyl acetate layer was dried over sodium sulfate and concentrated under vacuum at 40 to 45° C. Next, methanolic povidone USP (K29/32) solution (prepared by dissolving 1 gm povidone USP in 5 ml methanol) was added and stirred for 15 minutes. The resulted clear solution was concentrated to dryness using spray dryer (Jay Instruments & Systems Pvt. Ltd. India, Model-LSD-48 mini Spray Dryer). 4.35 g of a dried coprecipitate of atorvastatin calcium with povidone was obtained.

1. An amorphous co-precipitate comprising an atorvastatin pharmaceutically acceptable salt selected from atorvastatin magnesium or atorvastatin calcium and povidone (polyvinylpyrrolidone).

2-4. (canceled)

5. An The amorphous co-precipitate of claim 1, wherein:
a) when the amorphous co-precipitate comprises atorvastatin magnesium and povidone it is characterized by at least one of the following properties:
i) a powder X-ray diffraction pattern substantially in accordance with FIG. 1 or FIG. 3;
ii) an infra red (IR) spectrum substantially in accordance with FIG. 2 or FIG. 4;
iii) an infra red (IR) spectrum having absorption bands at about 3410, 2956, 1660, 1596, 1529, 1508, 1462, 1438, 1315, 1291, 1220, 1157, 845, 756 and 698±2_-cm⁻¹ substantially as depicted in FIG. 2; or
iv) an infra red (IR) spectrum having absorption bands at about 3403, 3057, 2957, 1660, 1595, 1527, 1509, 1461, 1435, 1312, 1289, 1221, 1156, 1076, 1031, 842, 753, 692±2 cm⁻¹ substantially as depicted in FIG. 4; and
v) a Scanning Electron Microscope (SEM) image of the morphological analysis in accordance with FIG. 5; and
b) when the amorphous co-precipitate comprises atorvastatin calcium and povidone it is characterized by at least one of the following properties:
i) a powder X-ray diffraction pattern substantially in accordance with FIG. 8 or Figure 9.
ii) an infra red (IR) spectrum substantially in accordance with FIG. 10; and
iii) an infra red (IR) spectrum having absorption bands at about 3412, 2924, 1660, 1595, 1508, 1462, 1438, 1315, 1290, 1219, 1156, 845, 756 and 696±2 cm⁻¹ substantially as depicted in FIG. 10.

6. (canceled)

7. A process for the preparation of the amorphous co-precipitate comprising atorvastatin pharmaceutically acceptable salt selected from atorvastatin magnesium or atorvastatin calcium and povidone claim 1, comprising the steps of:
a) condensing (4R-cis)-1,1-dimethylethyl 6-(2-aminoethyl)-2,2-dimethyl-1,3-dioxane-4-acetate of formula VI: 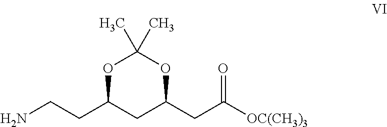
with (±)-4-fluoro-α-(2-methyl-1-oxopropyl)-γ-oxo-N,β-diphenylbenzenebutane amide of formula VII: 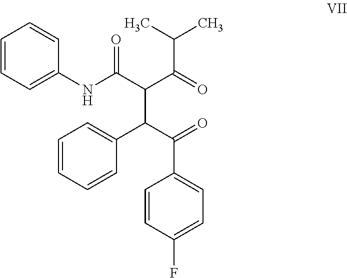
under acidic conditions to produce (4R-cis)-6-[2-[3-phenyl-4-(phenylcarbamoyl)-2-(4-fluorophenyl)-5(1-methylethyl)-pyrrol-1-yl]-ethyl]-2,2-dimethyl-[1,3]-dioxane-4-yl-acetic acid tertiary butyl ester of formula VIII: 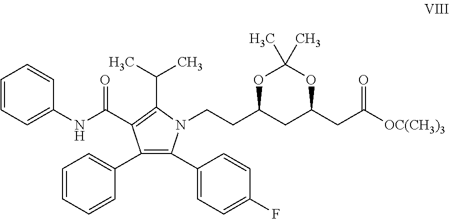
b) contacting the ester compound of formula VIII with an acid for acidic hydrolysis followed by treatment with a suitable base to produce atorvastatin sodium of formula IX: 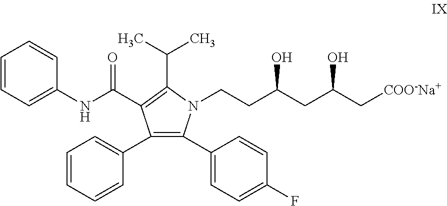
c) converting the atorvastatin sodium of formula IX into a pharmaceutically acceptable alkaline-earth metal salt of atorvastatin of formula II: 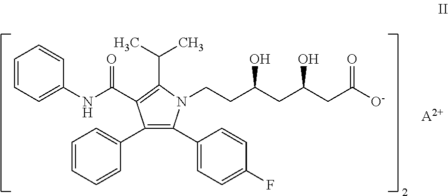
wherein ‘A’ represents an alkaline-earth metal selected from magnesium and calcium, in a water immiscible organic solvent by adding desired metal counter ion;
d) collecting the organic layer containing atorvastatin pharmaceutically acceptable salt obtained in step-(c);
e) evaporating partially the water immiscible organic solvent;
f) combining the reaction mass obtained in step-(e) with a homogeneous solution of a povidone in a solvent, wherein the solvent is an alcohol, a halogenated hydrocarbon, a ketone, an ester, a hydrocarbon, a nitrile, and mixtures thereof;
g) optionally, heating the reaction mixture obtained in step-(f);
h) removing the solvent from the solution obtained in step-(f) or step-(g);
i) optionally, drying the product obtained in step-(h); and
j) recovering the amorphous co-precipitate of atorvastatin pharmaceutically acceptable salt with povidone.

8. The process of claim 7, wherein the atorvastatin pharmaceutically acceptable salt formed in step-(c) is atorvastatin magnesium or atorvastatin calcium or a hydrate thereof; wherein when the salt is atorvastatin magnesium it is prepared by reacting atorvastatin sodium with a suitable magnesium source; and when the salt is atorvastatin calcium it is prepared by reacting atorvastatin sodium with a suitable calcium source.

9. (canceled)

10. The process of claim 98, wherein the magnesium source is an aqueous solution of magnesium hydroxide or a magnesium salt selected from the group consisting of magnesium acetate, magnesium chloride, magnesium sulphate, magnesium nitrate and magnesium phosphate; and wherein the calcium source is an organic or inorganic salt of calcium selected from the group consisting of calcium acetate, calcium proprionate, calcium butyrate, calcium tartrate, calcium benzoate, calcium phthalate, calcium stearate, calcium dodecanoate, calcium ascorbate, calcium succinate, calcium methane sulfonate, calcium benzene sulfonate, calcium p-toluene sulfonate, CaCl₂, CaF₂, CaBr₂ CaI₂, calcium borate (B₄CaO₇), calcium tetrafluoroborate (CaBF₄), calcium carbonate (CaCO₃), monobasic calcium phosphate (Ca(H₂PO₄)₂), dibasic calcium phosphate (CaHPO₄), tribasic calcium phosphate (Ca(PO₄)₂), calcium sulfate (CaSO₄), calcium hydroxide (Ca(OH)₂), and hydrates thereof.

11-16. (canceled)

17. The process of claim 10, wherein the calcium source is calcium acetate.

18. The process of claim 7, wherein the partial removal of the solvent in step-(e) is carried out by evaporation, atmospheric distillation, or distillation under vacuum wherein the combining in step-(f) is performed by dissolving povidone in a solution containing atorvastatin pharmaceutically acceptable salt and the solvent or dissolving atorvastatin pharmaceutically acceptable salt in a solution containing povidone and the solvent; and wherein the removal of the solvent in step-(h) is accomplished by complete evaporation of the solvent, spray drying, vacuum drying, lyophilization or freeze drying, or a combination thereof.

19-20. (canceled)

21. The process of claim 7, wherein the solvent used in step-(f) is selected from the group consisting of methanol, ethanol, isopropyl alcohol, n-propanol, dichloromethane, 1,2-dichloroethane, chloroform, carbon tetrachloride, acetone, ethyl methyl ketone, methyl isobutyl ketone, ethyl acetate, n-propyl acetate, n-butyl acetate, t-butyl acetate, toluene, xylene, acetonitrile, propionitrile, and mixtures thereof.

22-24. (canceled)

25. A process for the preparation of the amorphous co-precipitate comprising atorvastatin pharmaceutically acceptable salt selected from atorvastatin magnesium or atorvastatin calcium and povidone of claim 1, comprising the steps of:
a) providing a solution of comprising atorvastatin pharmaceutically acceptable salt and povidone in an organic solvent, wherein the atorvastatin pharmaceutically acceptable salt is atorvastatin magnesium or atorvastatin calcium, and wherein the organic solvent is an alcohol, a halogenated hydrocarbon, a ketone, an ester, a hydrocarbon, a nitrile, and mixtures thereof;
b) optionally, filtering the solvent solution to remove any extraneous matter; and
c) substantially removing the solvent from the solution to afford amorphous co-precipitates of atorvastatin pharmaceutically acceptable salt with povidone.

26-30. (canceled)

31. The process of claim 25, wherein the organic solvent used in step-(a) is selected from the group consisting of methanol, ethanol, isopropyl alcohol, n-propanol, dichloromethane, 1,2-dichloroethane, chloroform, carbon tetrachloride, acetone, ethyl methyl ketone, methyl isobutyl ketone, ethyl acetate, n-propyl acetate, n-butyl acetate, t-butyl acetate, toluene, xylene, acetonitrile, propionitrile, and mixtures thereof.

32. The process of claim 25, wherein the solution in step-(a) is prepared by dissolving any form of atorvastatin pharmaceutically acceptable salt in an organic solvent or obtained directly from a reaction in which atorvastatin pharmaceutically acceptable salts are formed, and then combining the solution with povidone; and wherein the removal of the solvent in step-(c) is accomplished by complete evaporation of the solvent, spray drying, vacuum drying, lyophilization or freeze drying, or a combination thereof.

33. The process of claim 32, wherein the dissolution is carried out at a temperature of about 20° C. to about 140° C.

34-35. (canceled)

36. A pharmaceutical composition comprising the amorphous co-precipitate of atorvastatin magnesium or atorvastatin calcium and povidone of claim 1, and a pharmaceutically acceptable excipient and wherein the pharmaceutical by a process comprising combining the amorphous co-precipitate of atorvastatin magnesium or atorvastatin calcium and providone with one or more pharmaceutically acceptable excipients.

37. (canceled)

38. A method for treating or preventing cardiovascular diseases caused by hypercholesterolemia, hyperlipoproteinemia, and atherosclerosis, comprising administering an amorphous co-precipitate of atorvastatin pharmaceutically acceptable salt selected from atorvastatin magnesium or atorvastatin calcium and povidone; or a pharmaceutical composition that comprises an amorphous co-precipitate of atorvastatin pharmaceutically acceptable salt selected from atorvastatin magnesium or atorvastatin calcium, and povidone, along with one or more pharmaceutically acceptable excipients.

39. The pharmaceutical composition of claim 36, wherein the amorphous co-precipitate of atorvastatin pharmaceutically acceptable salt with povidone has a D₉₀ particle size of less than or equal to about 500 microns.

40. The pharmaceutical composition of claim 39, wherein the 90 volume-percent of the particles (D₉₀) have a size of less than or equal to about 300 microns, less than or equal to about 200 microns, less than or equal to about 100 microns, or less than or equal to about 15 microns.

41-45. (canceled)
